(19) United States
(12) Patent Application Publication
(10) Pub. No.: US 2007/0250096 A1
(43) Pub. Date: Oct. 25, 2007

(54) Thrombectomy catheter

(75) Inventors: Masahisa Yamane, Tokyo (JP); Takenari Itou, Shizouka (JP); Tetsuya Fukuoka, Shizuoka (JP)

Correspondence Address: BUCHANAN, INGERSOLL & ROONEY PC, POST OFFICE BOX 1404, ALEXANDRIA, VA, 22313-1404, US

(73) Assignees: TERUMO KABUSHIKI KAISHA, Tokyo (JP); Masahisa YAMANE, Tokyo (JP)

(21) Appl. No.: 11/790,278

(22) Filed: Apr. 24, 2007

(30) Foreign Application Priority Data
Apr. 25, 2006 (JP) 2006-120571

(51) Int. Cl. A61B 17/22

(52) U.S. Cl. 606/159

(57) ABSTRACT

A catheter includes a flexible elongate catheter tube having a distal end, a proximal end, and a lumen defined therein, the proximal end being connectable to a suction device for evacuating the lumen, and a thrombus cutter disposed in the catheter tube near the distal end thereof, the thrombus cutter including at least one cutting edge directed radially inward from an inner wall surface of the lumen

### TECHNICAL FIELD

This invention generally relates to a catheter, and more particularly, to an aspiration thrombectomy catheter including a thrombus cutter.

### BACKGROUND OF THE INVENTION

Thrombectomy catheters have been used to continuously aspirate (i.e., draw) a blood clot from a blood vessel into the catheter lumen so as to remove the blood clot from the blood vessel. Specifically, after the tube of a thrombectomy catheter has been inserted through the blood vessel to a position where the thrombus is present, a suction device is connected to the proximal end of the catheter and activated to develop a negative pressure in the lumen of the tube and thereby remove the thrombus from the blood vessel.

One major problem that arises in using a thrombectomy catheter to remove a blood clot is that if the size (diameter) of the thrombus is greater than the inner diameter of the catheter lumen, the catheter lumen may be blocked by the thrombus.

When the lumen is blocked by the thrombus, the efficiency with which the thrombectomy catheter draws the clot is lowered possibly to the extent that the thrombectomy catheter may fail to draw the thrombus. One solution is to intensify the suction force (i.e., negative pressure) of the suction device to forcibly remove the blocking blood clot. However, since the blocking blood clot tends to be removed abruptly under the suction force, a temporarily excessive negative pressure is produced in the blood vessel, developing ischemia. If the blood vessel from which the thrombus is drawn is a narrow one, such as the coronary artery, then the blood vessel is liable to collapse under the excessive negative pressure. Furthermore, if a device such as a stent is placed in the blood vessel, then the device may be positionally displaced or deformed, e.g., crushed, under the excessive negative pressure.

In order to eliminate the blockage in the lumen caused by the blood clot, it is necessary to remove the thrombectomy catheter from the blood vessel. After the lumen blocked by the blood clot is cleaned with saline, the thrombectomy catheter is inserted again into the blood vessel. Alternatively, a new (fresh) thrombectomy catheter is inserted into the blood vessel. In either case, the blood vessel needs to be catheterized at least twice.

However, each time the thrombectomy catheter is inserted into the blood vessel, the patient feels pain. In order to reduce the danger of damage to the vessel wall, the thrombectomy catheter is moved very slowly and carefully through the blood vessel to the desired spot therein. As a result, it requires considerable time to remove the clot from the blood vessel. In addition, if the lumen blocked by the thrombus is cleaned with saline and then the thrombectomy catheter is once again inserted into the blood vessel, then additional time is required to clean the lumen.

Furthermore, when the cleaned thrombectomy catheter or a fresh thrombectomy catheter is to be inserted into the blood vessel, the position of a thrombus in the blood vessel has to be confirmed again.

If a fresh thrombectomy catheter is to be inserted into the blood vessel, then the fresh thrombectomy catheter needs to be completely sterilized as with the initially used thrombectomy catheter.

Various catheter designs are known in the art for solving the above problems in removing blood clots. For example, U.S. Pat. No. 4,646,736 discloses a device for removing a blood clot from a blood vessel through a lumen. The disclosed device has a rotational shaft for winding therearound the fibrin of the thrombus to fragment the thrombus and thereby allow the blood to again flow through the blood vessel. However, since the fibrin around the rotational shaft needs to be mechanically removed from the rotational shaft, it is necessary to pull the rotational shaft from the device.

Japanese Laid-Open Utility Model Publication No. Hei 2-61315 discloses a catheter device for removing a thrombus from a blood vessel. The disclosed catheter device has a rotational propeller on the distal end of a catheter for fragmenting the thrombus. When the lumen of the catheter is aspirated by a suction device, the thrombus that has been fragmented by the rotational propeller is continuously drawn out of the blood vessel.

However, the devices disclosed in U.S. Pat. No. 4,646,736 and Japanese Laid-Open Utility Model Publication No. Hei 2-61315 have many drawbacks. For example, the effective cross-sectional area of the lumen for drawing the thrombus therethrough is greatly reduced by the rotational shaft or the rotational propeller and a drive shaft for driving the rotational shaft or the rotational propeller. These devices are complex in structure and very expensive because of the rotational mechanism used therein.

The above disclosed devices in U.S. Pat. No. 4,646,736 and Japanese Laid-Open Utility Model Publication No. Hei 2-61315 generally include a disposable catheter part which is thrown away each time it has been used because it is inserted into the blood vessel, and a repetitively reusable drive unit such as a motor. The catheter part is very expensive as it has a complex rotational mechanism housed therein. The drive unit requires tedious and time-consuming work such as maintenance and the like in order to be repetitively reusable.

The disclosed devices have rigid distal ends on account of the rotational mechanism and the rotational shaft, and cannot be guided along curves of small radii of curvature. In addition, these devices have relatively large outside diameters. Consequently, the devices impose a limitation on the blood vessels in which they can be used.

To solve the problems of the above conventional devices, U.S. Pat. No. 5,569,204 reveals a thrombectomy catheter device for continuously aspirating a blood clot from a blood vessel. The thrombectomy catheter device has a central catheter housing therein an axially movable expander and an outer catheter disposed coaxially around the central catheter. The outer catheter has a distal end that can be pushed in up to the distal end of the central catheter.

If the lumen of the central catheter is blocked by a thrombus, then the central catheter is pulled out of the outer catheter. After the lumen of the central catheter has been cleaned, the central catheter is pushed back into the outer catheter. Alternatively, after the central catheter has been pulled out, a fresh central catheter is pushed into the outer catheter. Further alternatively, after the central catheter has been pulled out, a clot may be removed from the blood vessel through the outer catheter. Since the entire catheter assembly does not need to be removed from the blood vessel, the thrombotic removal can be resumed within a relatively short time.

However, the disclosed thrombectomy catheter device in U.S. Pat. No. 5,569,204 still requires the catheter suffering the thrombotic block, e.g., the central catheter or the outer catheter, to be removed out of the blood vessel.

Since the central catheter and the outer catheter provide a double-walled structure, the cross-sectional area that can be used for removing thrombus, of the overall cross-sectional area of the catheter assembly, is relatively small.

The double-walled structure of the central catheter and the outer catheter makes the rigidity of the catheter assembly relatively high. Therefore, when the thrombectomy catheter device is used with a tortuous blood vessel having small radii of curvature, it is difficult to insert the suction catheter device into the blood vessel.

The double-walled structure of the central catheter and the outer catheter also makes the operation of the thrombectomy catheter device relatively complex. For example, when the thrombectomy catheter device is in use, a hemostatic valve needs to be operated for each of the central catheter and the outer catheter. Furthermore, it is necessary that the central catheter and the outer catheter be operated in a desired positional relationship to each other, or specifically to bring their distal ends into an appropriate positional relationship to each other.

### SUMMARY

According to one aspect of the invention, these and other drawbacks are overcome by a catheter including a flexible elongate catheter tube having a distal end, a proximal end, and a lumen defined therein, the proximal end being connectable to suction means for evacuating the lumen, and a thrombus cutter disposed in the catheter tube near the distal end thereof.

The thrombus cutter may have a cutting edge extending in a direction from an inner wall surface of the lumen into the lumen.

The lumen may have a circular cross-sectional shape, and a straight line interconnecting the vertex of the cutting edge and the junction between the cutting edge and the inner wall surface of the lumen may be inclined to a line tangential to a circumferential surface of the lumen by an angle α in the range of 0°<α<90°.

The cutting edge may have a curved surface.

The thrombus cutter may include a hollow cylindrical tube of metal disposed in the catheter tube near the distal end thereof, and the cutting edge may include a wall portion of a circumferential wall of the hollow cylindrical tube, the wall portion being bent radially inwardly along a slit defined in the circumferential wall of the hollow cylindrical tube.

The slit may have a length in a circumferential direction of the hollow cylindrical tube, the length may be equal to or smaller than the diameter of the hollow cylindrical tube.

The length of the slit may be at least ⅛ of the diameter of the hollow cylindrical tube.

The angle θ at the center of the hollow cylindrical tube which subtends the arc of the slit and the angle r through which the cutting edge is bent from the circumferential wall of the hollow cylindrical tube may satisfy the following relationship:
10°≦r≦(180°−θ)/2

The cutting edge may have a distal end in a longitudinal direction of the hollow cylindrical tube, and the distance between the distal end of the cutting edge and the distal end of the catheter tube is in excess of 0 mm, but equal to or smaller than 3 mm.

The slit may have a length in a longitudinal direction of the hollow cylindrical tube, the length being in the range from 1 mm to 40 mm.

The length of the slit may be in the range from 5 mm to 20 mm.

When the lumen of the catheter is blocked by a thrombus in a blood vessel, the thrombus can easily be fragmented by the thrombus cutter and the blockage of the lumen can easily be eliminated in a simple operation, or specifically by manually applying a torque to the proximal end of the catheter tube to rotate the catheter tube about its longitudinal axis.

Therefore, the blocking by the thrombus of the lumen can quickly be eliminated and the operation to draw out and remove the thrombus can be quickly resumed.

When the lumen of the catheter is blocked by a thrombus in a blood vessel, it is not necessary to remove the catheter from the blood vessel and to introduce either the catheter which has been cleaned or a fresh catheter back into the blood vessel. Therefore, the time and labor required to remove the thrombus from the blood vessel are greatly reduced. The burden imposed on the patient when the thrombus is removed from the blood vessel is also greatly reduced.

The catheter is relatively simple in structure and can be manufactured relatively inexpensively because the thrombus which is blocking the lumen can be fragmented by manually rotating the catheter tube, without the need for a rotating mechanism.

As the catheter is free of a rotating mechanism, no maintenance of a drive unit which would be needed to actuate the rotating mechanism is required.

The catheter is thus free of other problems related to such a rotating mechanism. Specifically, the catheter does not suffer problems such as a reduction in the cross-sectional area of the lumen or a limitation on the range of blood vessels within which the catheter can be used, the limitation being imposed by an increase in the rigidity of the catheter, etc.

Furthermore, since the thrombus cutter is disposed in the catheter tube, the catheter will not cause damage to the inner wall surface of the blood vessel when the catheter tube is inserted into the blood vessel.

The above and other objects, features, and advantages of the present invention will become apparent from the following description when taken in conjunction with the accompanying drawings which illustrate preferred embodiments of the present invention by way of example.

### BRIEF DESCRIPTION OF THE DRAWING FIGURES

The foregoing and additional aspects of the disclosed device will become more apparent from the following detailed description considered with reference to the accompanying drawing figures briefly described below.

FIG. 1 is a side elevational view of a thrombectomy catheter, partly in cross section to show the internal structure of a distal end portion thereof;

FIG. 2A is an enlarged perspective view of a thrombus cutter disposed in the distal end of the thrombectomy catheter; FIG. 2B is an end view showing an open end of the thrombus cutter shown in FIG. 2A;

FIG. 3 is an enlarged perspective view of another thrombus cutter;

FIGS. 4A through 4D are end views similar to FIG. 2B showing the open end of the thrombus cutter shown in FIG. 3;

FIGS. 5A through 5E are end views similar to FIGS. 4A through 4D, showing still another thrombus cutter;

FIGS. 6A through 6E are end views similar to FIGS. 4A through 4D, showing yet another thrombus cutter;

FIGS. 7A through 7D are end views similar to FIGS. 4A through 4D, showing yet still another thrombus cutter;

FIGS. 8A and 8B are views similar to FIGS. 4A through 4D, showing yet still another thrombus cutter;

FIG. 9 is an enlarged fragmentary cross-sectional view of the distal end of a catheter tube of the thrombus cutter shown in FIGS. 2A and 2B;

FIG. 10 is a side elevational view which is illustrative of a procedure for using the thrombectomy catheter;

FIGS. 11A through 11H are development views of hollow cylindrical tubes of metal used in Inventive Examples; and

FIGS. 12A through 12H are development views of hollow cylindrical tubes of metal used in Inventive Examples.

### DETAILED DESCRIPTION

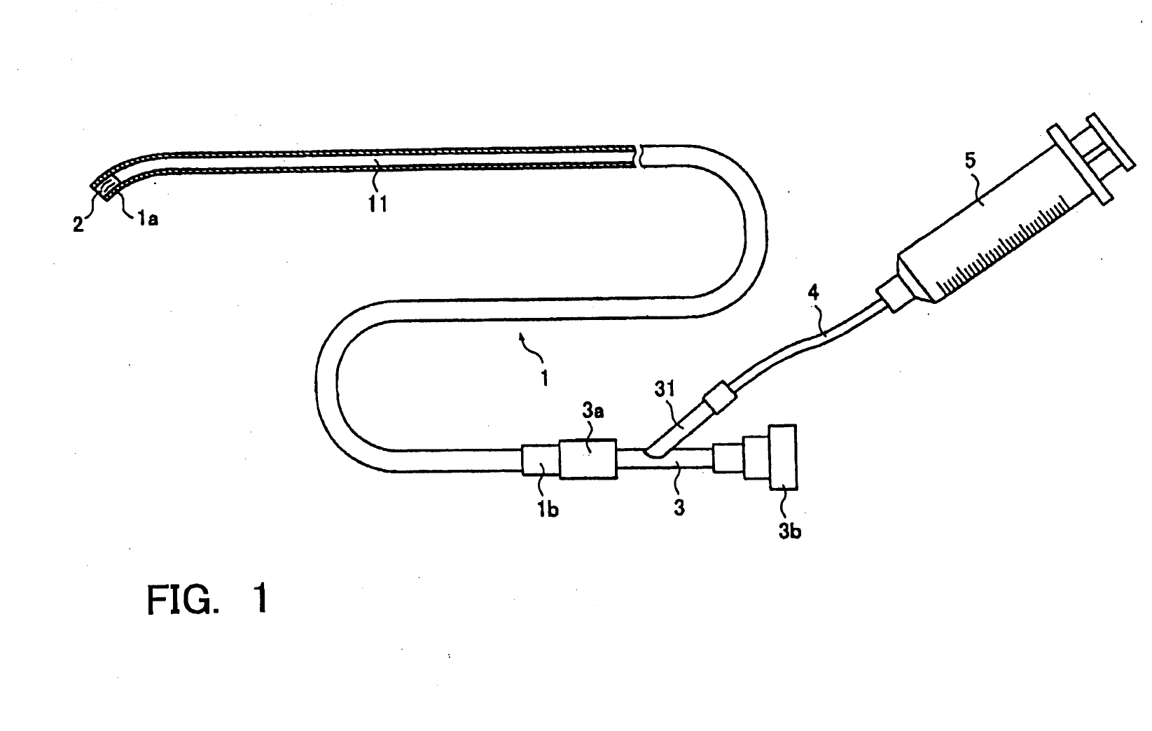

Referring to FIG. 1, an aspiration thrombectomy catheter assembly according to one illustrated and disclosed embodiment includes a flexible elongate catheter tube 1 having a lumen 11 defined therein. The lumen 11 has a circular cross-sectional shape. The thrombectomy catheter assembly also includes a catheter hub 1b mounted on the proximal end of the catheter tube 1, a Y-shaped connector 3 connected to the catheter hub 1b at the distal end 3a and a suction device (e.g., a syringe) 5 connected to a branch 31 of the Y-shaped connector 3 via a joint tube 4.

When the catheter tube 1 is inserted into a blood vessel, the end of the catheter tube 1 which is first introduced into the blood vessel is referred to as a distal end, and the other end of the catheter tube 1 as a proximal end. Elements other than the catheter tube 1 of the thrombectomy catheter assembly will also have distal and proximal ends defined according to the positional relationship between the distal and proximal ends of the catheter tube 1.

The suction device 5 should preferably include a syringe with a lock mechanism for locking the plunger in place while in operation to aspirate a thrombus. Alternatively, a three-way stopcock, not shown, may be provided on a distal or proximal end of the joint tube 4. The lock mechanism or the three-way stopcock makes it possible to keep negative pressure acting in the catheter tube 1 while the suction device 5 is in operation to draw a thrombus. When the thrombectomy catheter assembly is in use, a guide wire, not shown, is inserted from a proximal end 3b of the Y-shaped connector 3 into the lumen 11 in the catheter tube 1.

A thrombus cutter 2 is inserted in the lumen 11 of catheter tube 1 near its distal end 1a. When the lumen 11 in the catheter tube 1 is blocked by a thrombus, the operator manually applies a torque to the proximal end of the catheter tube 1 to turn the catheter tube 1 about its longitudinal axis, causing the thrombus cutter 2 to fragment the blocking thrombus. The thrombus cutter 2 has a plurality of cutting edges (i.e., cutting blades) for fragmenting the thrombus when the catheter tube 1 is turned about its longitudinal axis. The cutting edges do not necessarily have to be sharp cutting edges, like a knife, as long as they can fragment a thrombus which is generally soft, like agar or other gelatinous substances.

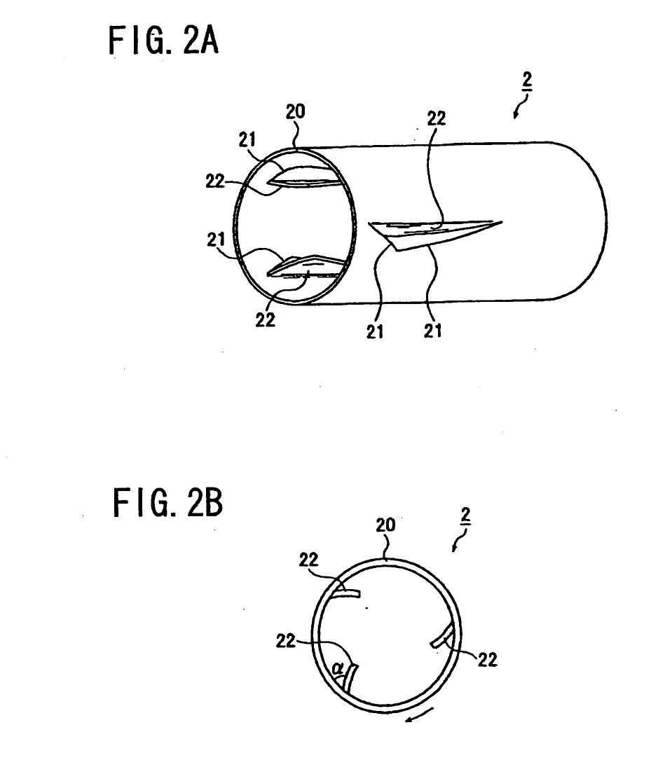

FIG. 2A shows the thrombus cutter 2 in enlarged perspective, and FIG. 2B shows a view from an open end of the thrombus cutter 2.

As shown in FIGS. 2A and 2B, the thrombus cutter 2 includes a hollow cylindrical tube 20 preferably made of metal and dimensioned to fit in the catheter tube 1. The hollow cylindrical tube 20 has a plurality of cutting edges 22 in the form of wall portions cut out along respective slits 21 defined in the cylindrical wall of the hollow cylindrical tube 20. The cutting edges 22 are bent radially inwardly from the cylindrical wall of the hollow cylindrical tube 20 and project in directions from an inner lumen wall surface of the catheter tube into the lumen in the catheter tube. In FIG. 2B, three cutting edges 22 project radially inwardly into the hollow cylindrical tube 20 as viewed from an open end of the hollow cylindrical tube 20.

The principles by which the thrombus cutter 2 cuts a thrombus will be described below.

When the catheter tube 1 shown in FIG. 1 is rotated about its longitudinal axis, the thrombus cutter 2 shown in FIG. 2B is rotated in the direction indicated by the arrow. The cutting edges 22 are bent around an axis parallel to the longitudinal direction of the catheter tube 1 from the cylindrical wall of the hollow cylindrical tube 20 through an acute angle with respect to the direction in which the thrombus cutter 2 rotates. Specifically, a straight line interconnecting the vertex of each of the cutting edges 22 and the base of the cutting edge 22, i.e., the bent corner of the cutting edge 22 which is joined to the hollow cylindrical tube 20, or the junction between the cutting edge 22 and the inner wall surface of the lumen 11 in the catheter tube 1, is inclined to a line tangential to the outer circumferential surface of the catheter tube 1 by an angle α (0°<α<90°). The direction in which the cutting edges 22 rotate is selected depending on the angle α, i.e., the cutting edges 22 are rotated in the direction in which the angle α is defined. Since the cutting edges 22 are bent from the cylindrical wall of the hollow cylindrical tube 20 through the acute angle with respect to the direction in which the thrombus cutter 2 rotates, the entry angle of the vertexes of the cutting edges 22 with respect to a thrombus blocking the lumen 11 is small. Therefore, the cutting edges 22 can easily bite into and break the thrombus, thereby efficiently cutting the thrombus.

Each of the cutting edges 22 should preferably have a curved surface having the same radius of curvature as the hollow cylindrical tube 20. The cutting edge 22 with a curved surface, particularly the curved surface having the same radius of curvature as the hollow cylindrical tube 20, is longer than a straight cutting edge provided the cutting edges have the same vertical height from the inner wall surface of the hollow cylindrical tube 20. Therefore, as the cutting edge 22 has a greater area of contact with the thrombus to be removed, the thrombus cutter 2 has a greater ability to remove the thrombus. At all points on the surface of the cutting edge 22, the cutting edge 22 is nearly parallel to the direction in which the thrombus cutter 2 rotates. Consequently, the angle of the cutting edge 22 with respect to the direction in which the thrombus cutter 2 rotates does not abruptly change while the thrombus cutter 2 is rotating. This feature is also preferable to increase the ability of the thrombus cutter 2 to remove the thrombus.

The thrombus cutter of the thrombectomy catheter according to the present invention is not limited to the hollow cylindrical tube of metal shown in FIGS. 2A and 2B as long as the thrombus cutter is positioned in the catheter tube near the distal end thereof when the thrombus cutter is inserted into the catheter tube near the distal end thereof, and the thrombus cutter has cutting edges for cutting a thrombus when the catheter tube is rotated about its longitudinal axis.

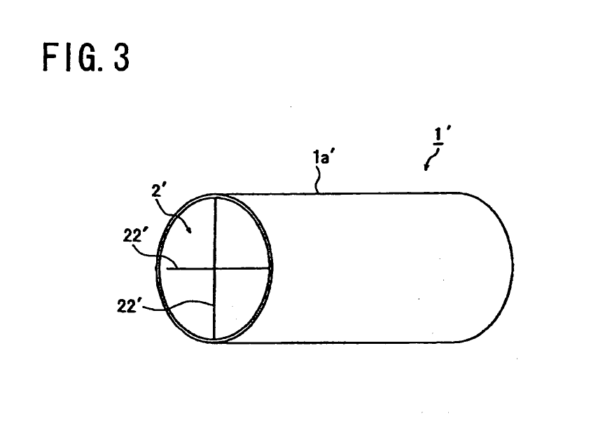

FIG. 3 shows a perspective view of another embodiment. As shown in FIG. 3, the thrombus cutter 2′ includes two ultrathin wires 22′ disposed in a crisscross pattern in the catheter tube 1′ near the distal end 1a′. The two ultrathin wires 22′ serve as cutting edges of the thrombus cutter 2′. The two ultrathin wires 22′ are so sharp that they can cut a thrombus at any angle. With the thrombus cutter 2′ shown in FIG. 3, the angle α referred to above may be 90°.

The cutting edges 22 shown in FIGS. 2A and 2B are easy to form and are less likely to obstruct the movement of a guide wire and a thrombus in the lumen 11 in the catheter tube 1. Therefore, the thrombus cutter 2 in the form of the hollow cylindrical tube 20 made of metal as shown in FIGS. 2A and 2B is preferable to the thrombus cutter 2′ shown in FIG. 3.

The cutting edges 22 are not limited to any particular shapes as long as they can cut a thrombus when the catheter tube 1 is rotated about its longitudinal axis. However, the thrombus cutter 2 shown in FIGS. 2A and 2B should preferably satisfy the following conditions.

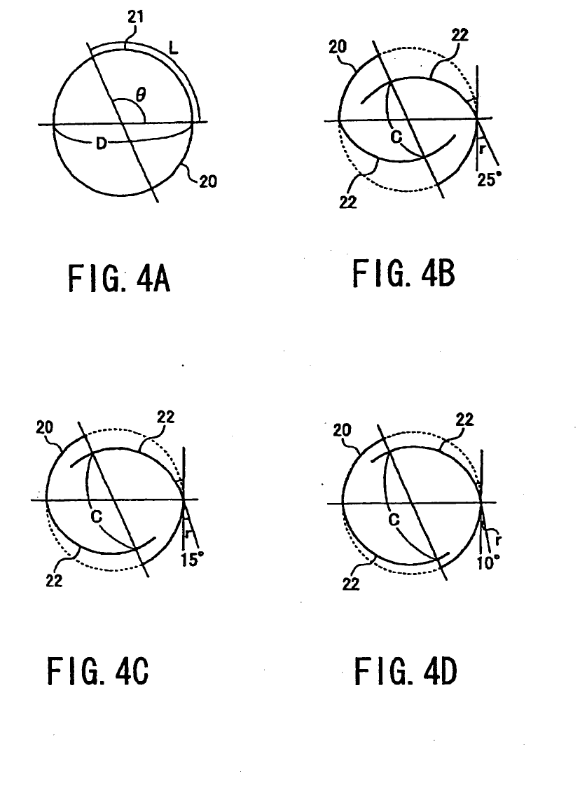

The conditions will be described below with reference to FIGS. 4A through 4D. FIGS. 4A through 4D are end views showing the open end of the thrombus cutter 2 as with FIG. 2B. FIG. 4A shows a tubular thrombus cutter blank before the wall portions are bent radially inwardly along the respective slits 21 defined in the cylindrical wall of the hollow cylindrical tube 20. FIGS. 4B through 4D show various angles at which the wall portions are bent radially inwardly along the respective slits 21 defined in the cylindrical wall of the hollow cylindrical tube 20 to form the cutting edges 22.

In FIG. 4A, the length L of the slit 21 in the circumferential direction of the hollow cylindrical tube 20 is preferably equal to or smaller than the diameter D of the hollow cylindrical tube 20. In FIG. 4A, D=2.0 mm and L=2.0 mm.

If the length L of the slit 21 in the circumferential direction of the hollow cylindrical tube 20 is equal to or smaller than the diameter D of the hollow cylindrical tube 20, then the length of the wall portion bent radially inwardly along the slit 21, i.e., the length of the cutting edge 22 in the circumferential direction of the hollow cylindrical tube 20, is not too large, so that the mechanical strength of the hollow cylindrical tube 20 is not unduly reduced. In addition, the cutting edge 22 does not project excessively into the hollow cylindrical tube 20, so that the cutting edge 22 will not obstruct the passage of a guide wire and a thrombus through the lumen 11 in the catheter tube 1.

The length L of the slit 21 in the circumferential direction of the hollow cylindrical tube 20 should preferably be at least ⅛ of the diameter D of the hollow cylindrical tube 20. With the length L thus selected, the cutting edge 22 projects sufficiently into the hollow cylindrical tube 20 to cut the thrombus.

The length L of the slit 21 in the circumferential direction of the hollow cylindrical tube 20 should preferably be in the range from ⅜ to ¾ of the diameter D of the hollow cylindrical tube 20.

The angle θ (see FIG. 4A) at the center of the hollow cylindrical tube 20 which subtends the arc of the slit 21 and the angle r (see FIGS. 4B through 4D) through which the cutting edge 22 is bent from the circumferential wall of the hollow cylindrical tube 20, should preferably satisfy the following relationship:
10°≦r≦(180°−θ)/2

The angle r through which the cutting edge 22 is bent from the circumferential direction of the hollow cylindrical tube 20 refers to an angle formed between a line tangential to the outer circumferential surface of the hollow cylindrical tube 20 at the base of the cutting edge 22, i.e., the region from which the wall portion of the cylindrical wall of the hollow cylindrical tube 20 is bent radially inwardly, and the radially inwardly bent wall portion of the cylindrical wall of the hollow cylindrical tube 20.

If the angle θ and the angle r satisfy the above relationship, then the cutting edge 22 projects sufficiently into the hollow cylindrical tube 20 to cut the thrombus, but does not project excessively into the hollow cylindrical tube 20, so that the cutting edge 22 will not obstruct the passage of a guide wire and a thrombus through the lumen 11 in the catheter tube 1.

The angle r should preferably be equal to or greater than 20°, but smaller than 90°, and more preferably in the range from 30° to 75°.

If the cutting edges of the thrombus cutter project forward from the distal end of the catheter tube, then the cutting edges tend to cause damage to the wall of the blood vessel when the catheter tube is moved to a desired spot in the blood vessel. Therefore, the cutting edges of the thrombus cutter should preferably not project forward from the distal end of the catheter tube.

The distance between the distal ends of the cutting edges 22 in the longitudinal direction of the hollow cylindrical tube 20 and the distal end 1a of the catheter tube 1 should preferably be in excess of 0 mm, but be equal to or smaller than 3 mm.

If the distance between the distal ends of the cutting edges 22 and the distal end 1a of the catheter tube 1 falls in the above range, then since the cutting edges 22 do not project forward from the distal end 1a of the catheter tube 1, the cutting edges 22 do not tend to cause damage to the wall of the blood vessel, and the position of the cutting edges 22 in the longitudinal direction of the catheter tube 1 is suitable for cutting the thrombus. The lumen 11 in the catheter tube 1 is blocked by the thrombus because the diameter of the thrombus is greater than the diameter of the lumen 11. Consequently, the portion of the catheter tube 1 near the distal end 1a thereof is liable to be blocked by the thrombus. If the distance between the distal ends of the cutting edges 22 and the distal end 1a of the catheter tube 1 falls in the above range, then since the cutting edges 22 are positioned near the distal end 1a of the catheter tube 1, the cutting edges 22 are suitable for cutting the thrombus that is blocking the lumen 11 in the catheter tube 1.

The distance between the distal ends of the cutting edges 22 in the longitudinal direction of the hollow cylindrical tube 20 and the distal end 1a of the catheter tube 1 should preferably be in the range from 0.1 mm to 2 mm.

The length of each of the slits 21 in the longitudinal direction of the hollow cylindrical tube 20 should preferably be in the range from 1 mm to 40 mm. If the length of each slit 21 is smaller than 1 mm, then the junction between the cutting edge 22 and the hollow cylindrical tube 20 is so weak that the cutting edge 22 may be broken off when the thrombectomy catheter is in use. If the length of each slit 21 is greater than 40 mm, then since the hollow cylindrical tube 20 itself is relatively long, it tends to make the catheter tube 1 inflexible near the distal end 1a thereof, so that the thrombectomy catheter can be used only with a limited range of blood vessels.

More preferably, the length of each of the slits 21 in the longitudinal direction of the hollow cylindrical tube 20 should be in the range from 5 mm to 20 mm.

If it is assumed that the cutting edges 22 are present in the thrombectomy catheter as shown in FIGS. 4B through 4D, then the clearance C (mm) between the cutting edges 22 near the center of the hollow cylindrical tube 20 and the diameter D_{g} (mm) of a guide wire, not shown, inserted through the catheter tube 1 should preferably satisfy the following equation:
C=D_{g}+0.1

The clearance C between the cutting edges 22 near the center of the hollow cylindrical tube 20 represents the length of a shortest straight line interconnecting the cutting edges 22 through the center of the hollow cylindrical tube 20. If the clearance C and the diameter D_{g} satisfy the above equation, then since clearance C between the cutting edges 22 near the center of the hollow cylindrical tube 20 is sufficiently larger than the diameter D_{g} of the guide wire inserted through the catheter tube 1, the guide wire will not be obstructed by the cutting edges 22 while it is in operation.

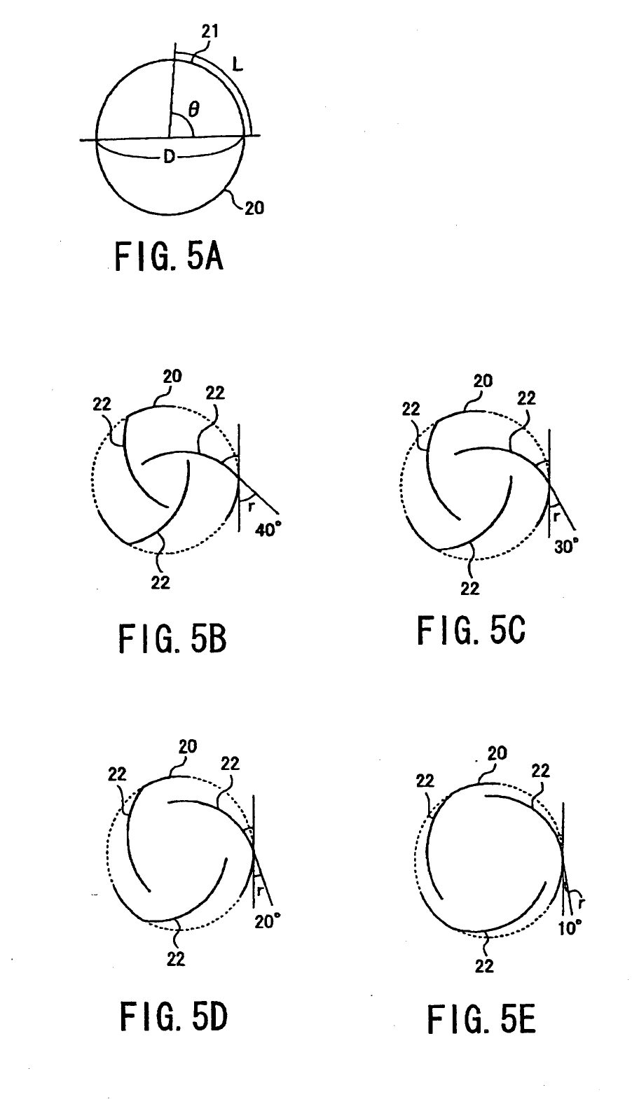

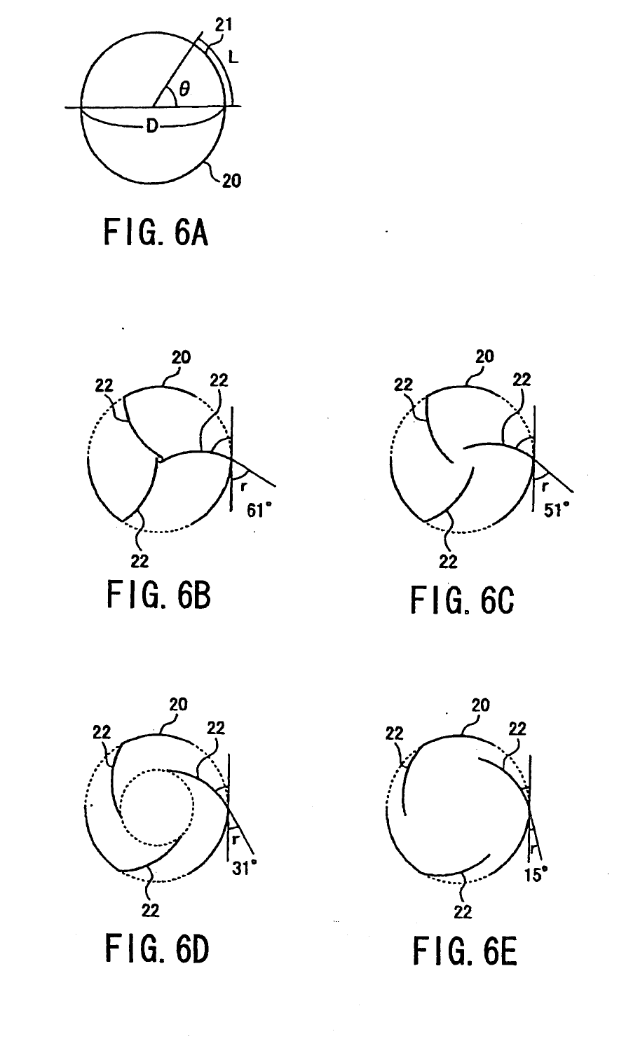

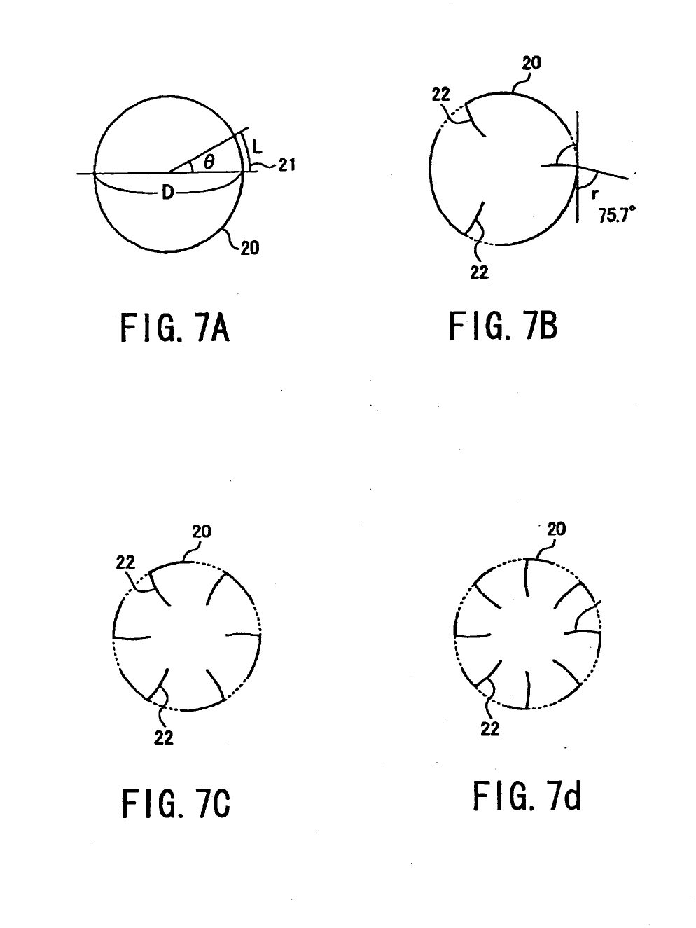

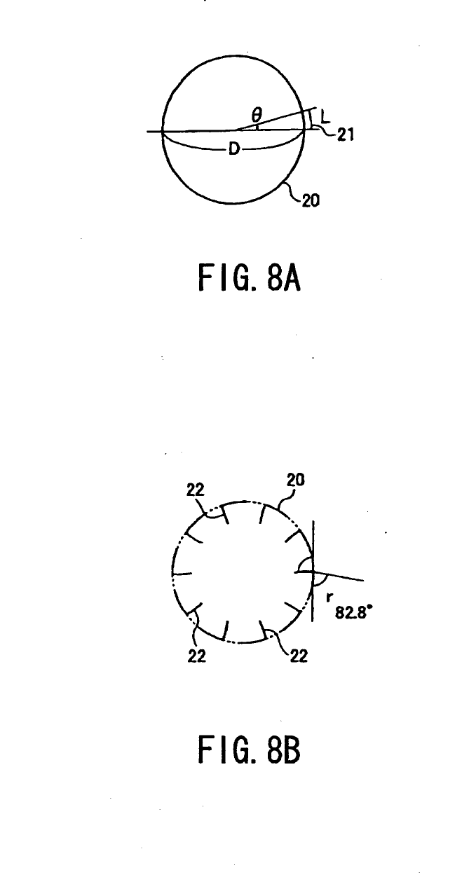

FIGS. 5A to 5E through 8A and 8B show various other thrombus cutters that can be used in the thrombectomy catheter according to an embodiment of the present invention. These thrombus cutters have different lengths L, different angles θ, different angles r, and different numbers of cutting edges 2 from those of the thrombus cutter shown in FIGS. 4A and 4B. In FIG. 5A, D=2.0 mm and L=1.5 mm. In FIG. 6A, D=2.0 mm and L=1.0 mm. In FIG. 7A, D=2.0 mm and L=0.5 mm. In FIG. 8A, D=2.0 mm and L=0.25 mm.

As can be seen from FIGS. 4A to 4D through 8A and 8B, the number of cutting edges of the thrombectomy cutter is not limited to any values. Therefore, the thrombectomy cutter may have a single cutting edge or a plurality of cutting edges in the circumferential direction of the hollow cylindrical tube 20. For a better thrombus cutting capability, the thrombectomy cutter should preferably have a plurality of cutting edges. If the thrombectomy cutter has a plurality of cutting edges, then the number of cutting edges should preferably be in the range from 2 to 8, and the cutting edges may be identical in shape to each other or different in shape from each other.

In FIGS. 4A to 4D through 8A and 8B, the thrombectomy cutter has a plurality of cutting edges spaced in the circumferential direction of the hollow cylindrical tube 20. However, the thrombectomy cutter may have a plurality of cutting edges spaced in the longitudinal direction of the hollow cylindrical tube 20. Preferably, the number of cutting edges in the longitudinal direction of the hollow cylindrical tube 20 should be in the range from 1 to 3.

If the thrombectomy cutter has a plurality of cutting edges spaced in the circumferential direction of the hollow cylindrical tube 20 as shown in FIGS. 4A to 4D through 8A and 8B, the cutting edges 22 may be positionally displaced in the longitudinal direction of the hollow cylindrical tube 20.

If the thrombectomy cutter has a plurality of cutting edges spaced in the longitudinal direction of the hollow cylindrical tube 20, or if the cutting edges 22 are positionally displaced in the longitudinal direction of the hollow cylindrical tube 20, the distance in the longitudinal direction of the hollow cylindrical tube 20 between the distal end of the cutting edge that is positioned most closely to the distal end of the hollow cylindrical tube 20 and the proximal end of the cutting edge that is positioned most closely to the proximal end of the hollow cylindrical tube 20 should preferably be in the range from 1 mm to 40 mm, or more preferably in the range from 5 mm to 20 mm.

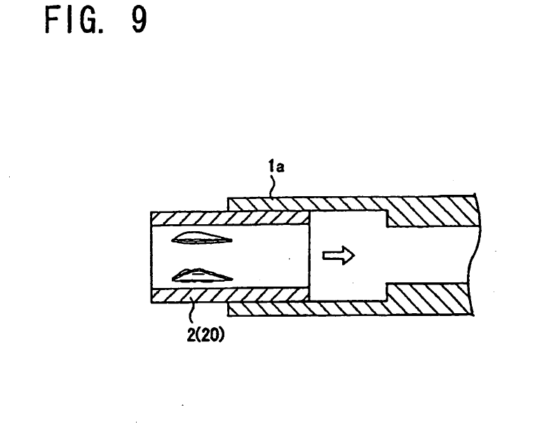

The dimensions of the hollow cylindrical tube 20 are selected depending on the dimensions of the catheter tube 1 through which the hollow cylindrical tube 20 is inserted. The hollow cylindrical tube 20 should preferably have an outside diameter which is substantially the same as the inside diameter of the catheter tube 1. If the outside diameter of the hollow cylindrical tube 20 is substantially the same as the inside diameter of the catheter tube 1, then the hollow cylindrical tube 20 can be placed in the catheter tube 1, and the hollow cylindrical tube 20 placed in the catheter tube 1 will not move in the catheter tube 1. However, as shown in FIG. 9, the hollow cylindrical tube 20 may have an outside diameter greater than the inside diameter of the catheter tube 1. FIG. 9 shows a portion of the catheter tube 1 near its distal end 1a. In FIG. 9, the outside diameter of the hollow cylindrical tube 20 is greater than the inside diameter of the catheter tube 1. In order for the catheter tube 1 to house the hollow cylindrical tube 20 therein, the inner wall surface of catheter tube 1 is enlarged in diameter near the distal end 1a thereof. The structure shown in FIG. 9 is preferable for preventing the hollow cylindrical tube 20 placed in the catheter tube 1 from moving toward the proximal end of the catheter tube 1.

The hollow cylindrical tube 20 may be secured in the catheter tube 1 by either adhesive bonding or thermal fusion. If adhesive bonding is used, then a preferable adhesive may be an olefinic adhesive, an acrylic adhesive, an epoxy adhesive, or an urethane adhesive. Of these adhesives, an epoxy adhesive is particularly preferable because it can provide sufficient bonding strength and the bonding strength is not lowered even when the bond is wet with water.

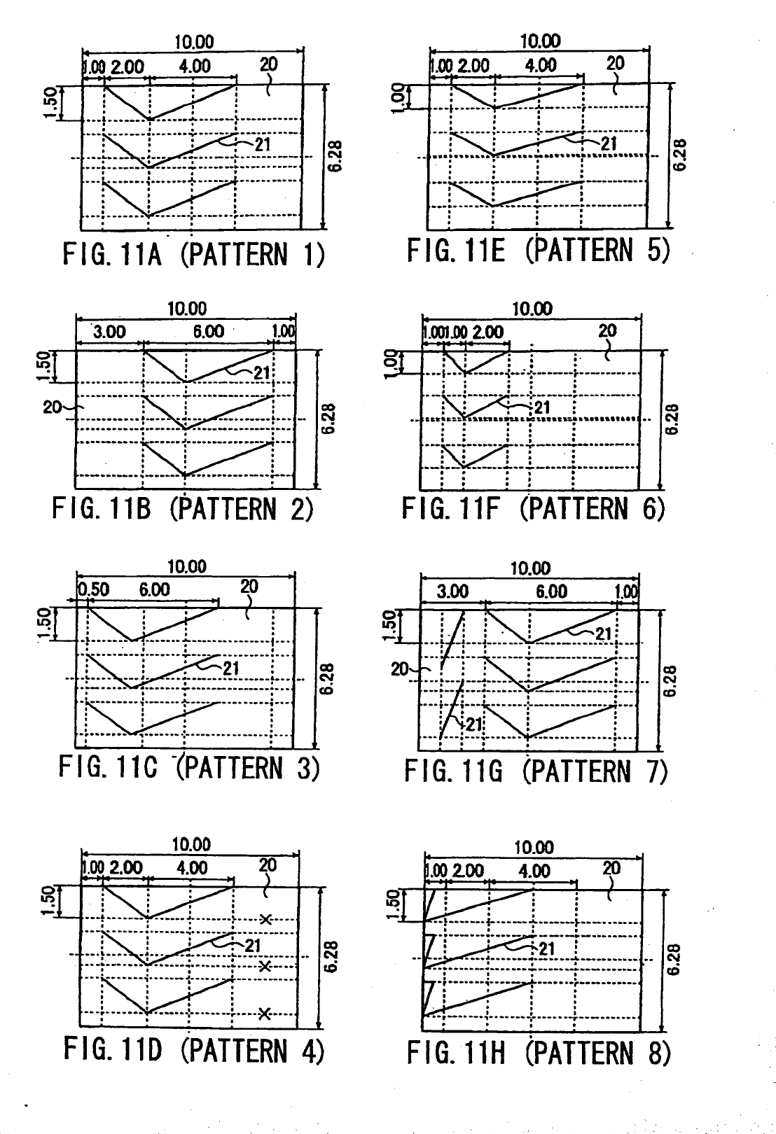

According to another securing scheme, several x-shaped slits are defined as securing protrusions in the hollow cylindrical tube 20, and after the hollow cylindrical tube 20 is inserted into the catheter tube 1 from its distal end, the slits are deformed so as to be spread outwardly from the inner surface of the hollow cylindrical tube 20, thereby securing the hollow cylindrical tube 20 in the catheter tube 1. These slits are shown in PATTERN 4 shown in FIG. 11D which is a development view of a hollow cylindrical tube of metal used in Inventive Example.

The length of the hollow cylindrical tube 20 should preferably be in the range from 3 mm to 45 mm. If the length of the hollow cylindrical tube 20 is smaller than 3 mm, then the cutting edges 22 that can be formed in the hollow cylindrical tube 20 are too small to cut the thrombus, or the junctions between the cutting edges 22 and the hollow cylindrical tube 20 are of insufficient strength, tending to allow the cutting edges 22 to be broken when the thrombectomy catheter is in use. If the length of the hollow cylindrical tube 20 is greater than 45 mm, then the catheter tube 1 tends to be inflexible near the distal end 1a thereof, so that the thrombectomy catheter can be used only with a limited range of blood vessels.

The length of the hollow cylindrical tube 20 should more preferably be in the range from 5 mm to 20 mm.

The wall thickness of the hollow cylindrical tube 20 should preferably be in the range from 0.03 mm to 0.3 mm. If the wall thickness of the hollow cylindrical tube 20 falls in the above range, then the hollow cylindrical tube 20 is of sufficient mechanical strength, and the wall thickness of the hollow cylindrical tube 20 is not too large to obstruct the passage of the thrombus through the lumen 11 or to make it difficult to form the slits 21 in the outer circumferential wall of the hollow cylindrical tube 20 to form the cutting edges 22.

The wall thickness of the hollow cylindrical tube 20 should more preferably be in the range from 0.05 mm to 0.2 mm.

The metal material of the hollow cylindrical tube 20 may be selected from metal materials that can be used for a device to be placed in blood vessels, such as a stent. Specific examples of these metal materials include stainless steel, tantalum, titanium, nickel titanium alloy, tantalum titanium alloy, nickel aluminum alloy, Inconel, gold, platinum, iridium, tungsten, cobalt-based alloy, etc. Of stainless steels, SUS316L or SUS304 which is of good corrosion resistance is preferable.

The outer circumferential wall of the hollow cylindrical tube of metal should preferably be slit by laser beam machining because the hollow cylindrical tube has very small dimensions. After the outer circumferential wall of the hollow cylindrical tube has been slit, wall portions of the outer circumferential wall of the hollow cylindrical tube can be bent inwardly along the slits by a punch or the like. In order to prevent the hollow cylindrical tube from being distorted, it is preferable that a core having holes which are defined therein at the bending positions and which are identical in shape to the cutting edges or slightly greater than the cutting edges be inserted into the hollow cylindrical tube, and then the wall portions be bent.

The core should preferably be made of ABS resin or polyester because it can be dissolved away using a solvent after the cutting edges have been formed. If the core is dissolved away using a solvent, then the formed cutting edges do not tend to be damaged, and core residuals around the cutting edges do not tend to remain unremoved. A core having a desired shape can be obtained by laser beam machining or injection molding.

The outside diameter of the catheter tube 1 should preferably be in the range from about 1.0 to 3.0 mm, and more preferably in the range from about 1.4 to 2.7 mm. The inside diameter of the catheter tube 1 should preferably be in the range from about 0.5 to 2.7 mm, and more preferably in the range from about 1.1 to 2.4 mm. The length of the catheter tube 1 should preferably be in the range from about 500 to 2000 mm, and more preferably in the range from about 800 to 1500 mm.

The catheter tube 1 is made of, for example, polyolefin such as polypropylene, polyethylene, or the like, or olefinic elastomer (e.g., polyethylene elastomer or polypropylene elastomer), or polyester such as polyethylene terephthalate or polyester elastomer, soft polyvinyl chloride, polyurethane, or urethane elastomer, polyamide or amide elastomer (e.g., polyamide elastomer), polytetrafluoroethylene or fluororesin elastomer, or a pliable polymer material such as polyimide, ethylene—vinyl acetate copolymer, silicone rubber, or the like.

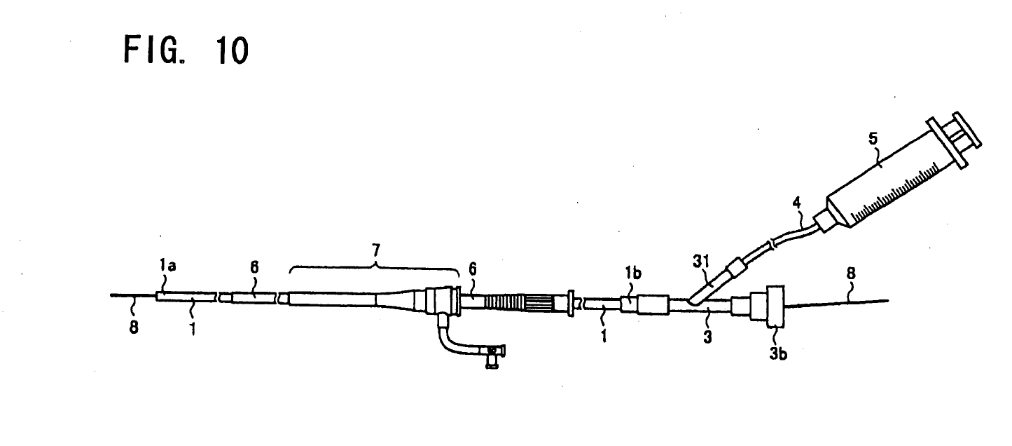

A procedure for using the thrombectomy catheter according to an embodiment of the present invention will be described below with reference to FIG. 10. The thrombectomy catheter, by way of example, corresponds to the embodiment shown in FIG. 1.

For using the thrombectomy catheter, a guide wire 8 is first inserted into the blood vessel according to the Seldinger method, and then an introducer sheath 7 is inserted into the blood vessel. Then, a guiding catheter 6 is introduced along the guide wire 8 into the blood vessel from which the thrombus is to be removed. Finally, the thrombectomy catheter (catheter tube) 1 is introduced along the guiding catheter 6 until the distal end 1a of the catheter tube 1 is placed in a region from which the thrombus is to be removed.

Then, the fluid passage in the proximal end 3b of the Y-shaped connector 3 that is connected to the catheter hub 1b of the catheter tube 1 is closed by a valve, not shown, connected to the proximal end 3b. Thereafter, the lumen 1 in the catheter tube 1 is evacuated by the suction device 5, drawing the thrombus in the blood vessel. At this time, the thrombus may be drawn while the catheter tube 1 is being rotated about its longitudinal axis by a manually applied torque.

If the lumen 11 in the catheter tube 1 is blocked by the thrombus, the evacuating operation of the suction device 5 is stopped, and the catheter hub 1b of the catheter tube 1 is held by hand. A torque is manually applied to rotate the catheter tube 1 about its longitudinal axis. As described above, when the catheter tube 1 is rotated about its longitudinal axis, the cutting edges 22 of the thrombus cutter 2 fragment the thrombus which is blocking the lumen 11.

Therefore, even if the lumen 11 in the catheter tube 1 is blocked by the thrombus, the thrombus can easily be fragmented by a simple process of rotating the catheter tube 1 about its longitudinal axis. Therefore, the blocking by the thrombus of the lumen 11 can quickly be eliminated and the operation of drawing out the thrombus can be quickly resumed.

The thrombectomy catheter according to one preferred embodiment has been described above with reference to the drawings. However, the thrombectomy catheter according to the present invention is not limited to the above illustrated embodiments. The suction device is not limited to the syringe, but may be another vacuum producing means such as a pump or the like. The outer surface of the catheter tube 1 near its proximal end or the outer surface of the catheter hub 1b may be marked with an arrow or the like indicative of the preferred direction in which to rotate the thrombus cutter 2. The preferred direction in which to rotate the thrombus cutter 2 refers to a direction with respect to which the cutting edges 22 are bent through an acute angle, such as the direction indicated by the arrow in FIG. 2B.

### INVENTIVE EXAMPLE

Inventive Examples of the present invention will be described below.

In the Inventive Examples, thrombus cutters 2 as shown in FIGS. 2A and 2B were produced from hollow cylindrical tubes of stainless steel (having an outside diameter of 2.0 mm, an inside diameter of 1.8 mm, and lengths of 5, 10, and 12 cm). Slits 21 were formed in portions of the outer circumferential walls of the hollow cylindrical tubes 20 by a laser beam, and wall portions of the outer circumferential walls of the hollow cylindrical tubes 20 were bent inwardly along the slits 21, thereby forming thrombus cutters 2 having cutting edges 22.

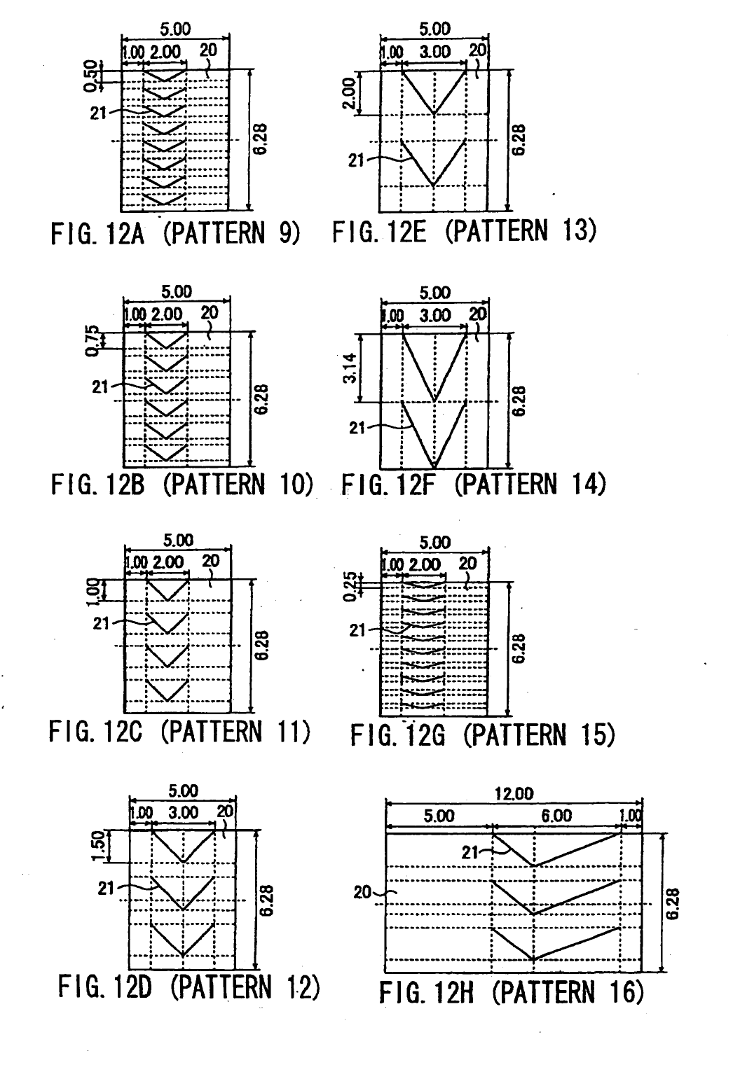

FIGS. 11A through 11H and 12A through 12H are development views of the hollow cylindrical tubes of metal used in Inventive Examples, showing the shapes and positions of the slits 21. In FIGS. 11A through 11H and 12A through 12H, the left side represents a distal end side and the right side a proximal end side. All the numerical values shown in FIGS. 11A through 11H and 12A through 12H have a unit of mm. In PATTERN 4 shown in FIG. 11D, the hollow cylindrical tube 20 has three small x-shaped slits defined as protrusions for securing the hollow cylindrical tube 20. After the hollow cylindrical tube 20 is inserted into the catheter tube 1 from its distal end, the slits are deformed so as to be spread outwardly from the inner surface of the hollow cylindrical tube 20, thereby securing the hollow cylindrical tube 20 in the catheter tube 1.

Each of the thrombus cutters 2 fabricated according to the above process was inserted into the catheter tube (having an inside diameter of 2.06 mm). The distance between the distal end of the inserted thrombus cutter 2 and the distal end of the catheter tube 1 was 1 mm.

The proximal end of the catheter tube 1 was connected to the Y-shaped connector 3 by the catheter hub lb. The suction device (a syringe with a lock mechanism) was connected to the branch 31 of the Y-shaped connector 3 by the joint tube 4 and the three-way stopcock, not shown. The proximal end 3b of the Y-shaped connector 3 was hermetically sealed.

After the syringe 5 was pulled to develop a negative pressure therein, the three-way stopcock was opened to introduce the negative pressure into the catheter tube 1. Then, while the catheter tube 1 was being rotated about its longitudinal axis, the distal end 1a of the catheter tube 1 was inserted into an agar gel to the depth of 30 mm.

After the catheter tube 1 was pulled out of the agar gel, the distal end 1a of the catheter tube 1 was dipped in water. Then, the syringe 5 was operated to draw the agar gel, together with water, which had been introduced into the catheter tube 1, thereby retrieving the agar gel. The retrieved agar gel was filtered by a filter, and thereafter observed for its shape. The same process was conducted on a specimen wherein the thrombus cutter was not inserted into the catheter tube 1 (Comparative Example 1) and a specimen wherein a hollow cylindrical tube of metal that was free of cutting edges was inserted into the catheter tube 1 (Comparative Example 2). The results are shown in the table below.

TABLE 1

| | State of catheter tube |
|---|---|
| Inventive Example 1 | Not closed |
| Inventive Example 2 | Not closed |
| Inventive Example 3 | Not closed |
| Inventive Example 4 | Not closed |
| Inventive Example 5 | Not closed |
| Inventive Example 6 | Not closed |
| Inventive Example 7 | Not closed |
| Inventive Example 8 | Not closed |
| Inventive Example 9 | Not closed |
| Inventive Example 10 | Not closed |
| Inventive Example 11 | Not closed |
| Inventive Example 12 | Not closed |
| Inventive Example 13 | Not closed |
| Inventive Example 14 | Closed but not completely |
| Inventive Example 15 | Closed but not completely |
| Inventive Example 16 | Closed but not completely |
| Comparative Example 1 | Closed |
| Comparative Example 2 | Closed |

In the table, the symbols signify the following results:

Not closed: The catheter tube was not closed by the agar gel. The retrieved agar gel was finely fragmented.

Closed but not completely: Though the catheter tube was closed by the agar gel, the blockage was eliminated when the suction force from the syringe was increased. The retrieved agar gel was not substantially fragmented.

Closed: The catheter tube was closed by the agar gel. The blockage was not eliminated even when the suction force from the syringe was increased. The retrieved agar gel was essentially not fragmented.

Although certain preferred embodiments of the present invention have been shown and described in detail, it should be understood that various changes and modifications may be made therein without departing from the scope of the appended claims.

What is claimed is:

1. A catheter comprising:
a flexible elongate catheter tube having a distal end, a proximal end, and a lumen defined therein, the proximal end being connectable to suction means for evacuating the lumen;
a thrombus cutter disposed within said catheter tube near the distal end thereof, said thrombus cutter including at least one cutting edge directed radially inward from an inner wall surface of the lumen.

2. The catheter according to claim 1, wherein the lumen has a circular cross-sectional shape, and a straight line interconnecting the vertex of the cutting edge and the junction between the cutting edge and the inner wall surface of the lumen is inclined to a line tangential to a circumferential surface of the lumen by an angle α in the range of 0°<α<90°.

3. The catheter according to claim 1, wherein the cutting edge has a curved surface.

4. The catheter according to claim 1, wherein the thrombus cutter includes a hollow cylindrical tube of metal disposed in the catheter tube near the distal end thereof, and the cutting edge includes a wall portion of a circumferential wall of the hollow cylindrical tube, the wall portion being bent radially inwardly along a slit defined in the circumferential wall of the hollow cylindrical tube.

5. The catheter according to claim 4, wherein the slit has a length in a circumferential direction of the hollow cylindrical tube, the length being equal to or smaller than the diameter of the hollow cylindrical tube.

6. The catheter according to claim 5, wherein the length of the slit is at least ⅛ of the diameter of the hollow cylindrical tube.

7. The catheter according to claim 6, wherein an angle θ at the center of the hollow cylindrical tube which subtends the arc of the slit and an angle r through which the cutting edge is bent from the circumferential wall of the hollow cylindrical tube satisfy the following relationship:
10°≦r≦(180°−θ0)/2

8. The catheter according to claim 7, wherein the cutting edge has a distal end in a longitudinal direction of the hollow cylindrical tube, and the distance between the distal end of the cutting edge and the distal end of the catheter tube is in excess of 0 mm, but equal to or smaller than 3 mm.

9. The catheter according to claim 8, wherein the slit has a length in a longitudinal direction of the hollow cylindrical tube, the length being in the range from 1 mm to 40 mm.

10. The catheter according to claim 9, wherein the length of the slit is in the range from 5 mm to 20 mm.

11. The catheter according to claim 4, wherein the cutting edge has a distal end in a longitudinal direction of the hollow cylindrical tube, and the distance between the distal end of the cutting edge and the distal end of the catheter tube is in excess of 0 mm, but equal to or smaller than 3 mm.

12. The catheter according to claim 4, wherein the slit has a length in a longitudinal direction of the hollow cylindrical tube, the length being in the range from 1 mm to 40 mm.

13. A thrombectomy catheter comprising:
a flexible elongate catheter tube having a distal end, a proximal end, and a lumen defined therein, the proximal end being connectable to suction means for evacuating the lumen;
a thrombus cutter-assembly including a tubular portion and at least one blade portion, said cutter assembly being disposed within said catheter tube near the distal end thereof, said blade portion extending radially inward from an inner wall surface of said tubular portion of said cutter assembly.

14. The catheter according to claim 13, wherein said at least one blade portion defines a cutting edge.

15. The catheter according to claim 14, wherein the lumen has a circular cross-sectional shape, and a straight line interconnecting the vertex of the cutting edge and the junction between the cutting edge and an inner wall surface of the lumen is inclined to a line tangential to a circumferential surface of the lumen by an angle α in the range of 0°<α<90°.

16. The catheter according to claim 13, wherein the cutting edge has a curved surface.

17. The catheter according to claim 13, wherein the thrombus cutter assembly includes a hollow cylindrical tube defining said tubular portion, said tubular portion having a slit, and wherein said blade portion is defined by a wall portion of the hollow cylindrical tube, the wall portion being bent radially inwardly along the slit defined in a circumferential wall of the tubular portion.

18. The catheter according to claim 17, wherein the slit has a length in a circumferential direction of the hollow cylindrical tube, the length being equal to or smaller than the diameter of the hollow cylindrical tube.

19. The catheter according to claim 18, wherein the length of the slit is at least ⅛ of a diameter of the hollow cylindrical tube.

20. The catheter according to claim 17, wherein an angle θ at the center of the hollow cylindrical tube which subtends the arc of the slit and an angle r through which the cutting edge is bent from the circumferential wall of the hollow cylindrical tube satisfy the following relationship:
10°≦r≦(180°−θ)/2
